SEQUENCE LISTING (1 sequence)

SEQ ID NO: 1 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

(19) United States
(12) Patent Application Publication
(10) Pub. No.: US 2025/0114343 A1
(43) Pub. Date: Apr. 10, 2025

(54) Treatment of Bronchiectasis

(71) Applicants: Emory University, Atlanta, GA (US); Children's Healthcare of Atlanta, Inc., Atlanta, GA (US)

(72) Inventors: Eric J. Sorscher, Atlanta, GA (US); Rachel W. Linnemann, Atlanta, GA (US); Arlene A. Stecenko, Atlanta, GA (US); William R. Hunt, Atlanta, GA (US)

(21) Appl. No.: 18/730,046

(22) PCT Filed: Jan. 18, 2023
(86) PCT No.: PCT/US2023/060828

§ 371 Date: Jul. 18, 2024

Related U.S. Application Data
(60) Provisional application No. 63/300,373, filed on Jan. 18, 2022.

(51) Int. Cl. A61K 31/47; A61K 31/404; A61K 31/443; A61K 45/06; A61P 11/00

(52) U.S. Cl. CPC A61K 31/47; A61K 31/404; A61K 31/443; A61K 45/06; A61P 11/00

(57) ABSTRACT

This disclosure relates to methods of treating bronchiectasis comprising administering an effective amount of a cystic fibrosis drug to a subject. In certain embodiments, the subject is diagnosed with non-CF bronchiectasis and the subject is diagnosed with moderate elevated sweat chloride and optionally pancreatic sufficiency. In certain embodiments, a sample of the subject is tested for presence of a known cystic fibrosis transmembrane conductance regulator mutation and no mutation is identified in the sample, thereby providing a subject diagnosed without a known cystic fibrosis transmembrane conductance regulator mutation. In certain embodiments, this disclosure relates to methods of bronchiectasis treatment by managing symptoms such as slowing decline in lung function and preventing exacerbations.

### CROSS-REFERENCE TO RELATED APPLICATIONS

This application claims the benefit of U.S. Provisional Application No. 63/300,373 filed Jan. 18, 2022. The entirety of this application is hereby incorporated by reference for all purposes.

### STATEMENT REGARDING FEDERALLY SPONSORED RESEARCH OR DEVELOPMENT

This invention was made with government support under grant HL139876 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 22005PCT.xml. The XML file is 4 KB, was created on Jan. 17, 2023, and is being submitted electronically via the USPTO patent electronic filing system.

### BACKGROUND

Bronchiectasis occurs in the lungs when the airway walls become thickened and scarred preventing the bronchi from effectively draining normal secretions creating mucus at risk of harboring bacterial infections. Cycles of inflammation and infection create irregular airway pockets leading to lung deterioration. Bronchiectasis is common in the inherited disease, cystic fibrosis (CF). Disease-associated variants are contained in the CF transmembrane conductance regulator (CFTR) gene, which encodes an epithelial cell ion channel that is defective in patients with CF. Failure of the CFTR ion channel causes inflammation, chronic infection, and fibrotic scarring of the respiratory parenchyma. This condition also affects tissues in other organs such as the pancreas and liver.

Ivacaftor is a “potentiator”-type modulator (a drug that helps open the CFTR ion channel gate) and improves lung function. The drug has gained U.S. Food and Drug Administration (FDA) approval when a patient is diagnosed with certain CFTR mutations. In addition, ivacaftor in combination with lumacaftor (a “corrector” of decreased CFTR biogenesis) is approved for individuals with two copies of the class II Phe508del CFTR protein maturation abnormality—the most common CFTR mutation.

Manfredi et al. report making precision medicine personal for cystic fibrosis. Science. 2019, 365(6450): 220-221.

Patel et al. report CFTR targeted therapies and possibilities in other diseases of the airways. Eur Respir Rev, 2020, 29: 190068.

Shaughnessy et al. report elexacaftor is a CFTR potentiator that acts synergistically with ivacaftor during acute and chronic treatment. Scientific Reports, 2021, 11:1.

Bratcher et al. report methods of treating cystic fibrosis transmembrane conductance regulator (CFTR)-mediated disease, such as cystic fibrosis, in patients with residual function mutations. See U.S. Patent Application Publication No. 2021/0196728.

References cited herein are not an admission of prior art.

### SUMMARY

This disclosure relates to methods of treating bronchiectasis comprising administering an effective amount of a cystic fibrosis drug to a subject. In certain embodiments, the subject is diagnosed with bronchiectasis and the subject is diagnosed with moderate elevated sweat chloride and optionally pancreatic sufficiency. In certain embodiments, a sample of the subject is tested for presence of a known cystic fibrosis transmembrane conductance regulator mutation and no mutation is identified in the sample, thereby providing a subject diagnosed without a known cystic fibrosis transmembrane conductance regulator mutation. In certain embodiments, this disclosure relates to methods of bronchiectasis treatment by managing symptoms such as slowing decline in lung function and preventing exacerbations.

In certain embodiments, this disclosure contemplates treating subjects, e.g., human patients with a diagnosis of non-cystic fibrosis bronchiectasis, e.g., due to moderate elevated sweat chloride, lack of the ability to identify a known cystic fibrosis transmembrane conductance regulator mutation, pancreatic sufficiency or combinations thereof, that respond to treatment with effective amounts of cystic fibrosis drugs. In certain embodiments, the cystic fibrosis drug is lumacaftor, elexacaftor, ivacaftor, tezacaftor, or combinations thereof. In certain embodiments, the cystic fibrosis drug is a combination of elexacaftor, ivacaftor, and tezacaftor. In certain embodiments, the cystic fibrosis drug is a combination of ivacaftor and lumacaftor. In certain embodiments, the cystic fibrosis drug is a combination of ivacaftor and tezacaftor.

In certain embodiments, this disclosure contemplates a non-personalized use of CF modulators in patients with non-CF bronchiectasis, e.g., those with sweat chloride levels of between 30 to 60 mEq/L and without a diagnosis of a clinically approved genotype.

In certain embodiments, this disclosure contemplates uses in treating patients without disease-causing CFTR mutations having bronchiectasis using a treatment with CFTR modulators as reported herein.

In certain embodiments, this disclosure contemplates uses in treating patients with non-CF bronchiectasis and a mildly elevated sweat chloride (i.e., suggesting diminished CFTR activity) not sufficient to establish a diagnosis of CF (but instead leading to a diagnosis of non-CF bronchiectasis) using a treatment with cystic fibrosis drugs as reported herein.

In certain embodiments, this disclosure contemplates uses in treating patients with non-CF bronchiectasis and normal sweat chloride (normal CFTR function in sweat glands) using a treatment with cystic fibrosis drugs as reported herein wherein activating the endogenous (and non-mutant) CFTR enhances mucus clearance and/or minimizes airway remodeling damage.

In certain embodiments, the subject is diagnosed with cylindrical bronchiectasis. In certain embodiments, the subject is diagnosed with varicose bronchiectasis or cystic bronchiectasis not associated with cystic fibrosis.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims. “Embodiments” refer to an example, and it is contemplated that the embodiments are not necessarily limited to the example.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent. Also, the term “or” is used in its inclusive sense (and not in its exclusive sense) so that when used, for example, to connect a list of elements, the term “or” means one, some, or all of the elements in the list.

As used herein, the term “about” is synonymous with the term “approximately.” Illustratively, the use of the term “about” indicates that a value includes values slightly outside the cited values. Variation may be due to conditions such as experimental error, manufacturing tolerances, variations in equilibrium conditions, and the like. In some embodiments, the term “about” includes the cited value plus or minus 10%. In all cases, where the term “about” has been used to describe a value, it should be appreciated that this disclosure also supports the exact value.

The terms “comprising,” “including,” “having,” and the like are synonymous and are used inclusively, in an open-ended fashion, and do not exclude additional elements, features, acts, operations, and so forth.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods, compared to those of the corresponding compositions or methods disclosed herein.

“Subject” refers to any animal, preferably a human patient, livestock, rodent, monkey, or domestic pet.

“Bronchiectasis” refers to a condition where the walls of the bronchi are thickened from inflammation or other causes which can result in periodic flare-ups of breathing difficulties, also referred to as exacerbations. Cylindrical (tubular) bronchiectasis is characterized by cylinder-shaped bronchi/bronchioles. Cylindrical bronchiectasis is a morphologic type of bronchiectasis where there is smooth uniform enlargement of bronchi with loss of the normal distal tapering of the airways without focal outpouchings. Bronchial dilatation is typically evaluated in relation to the accompanying pulmonary artery. A broncho to arterial ratio greater than 1:1 is typically considered abnormal. Normal bronchi are narrower in diameter the further they are from the lung hilum. Lack of normal bronchial tapering over 2 cm in length, distal from an airway bifurcation, is a sign of bronchiectasis. Varicose bronchiectasis bronchi are irregular, and the airways may be wide or constricted. In cystic bronchiectasis, cysts can occur in the subpleural areas, when they typically represent paraseptal emphysema, bullae, or honeycombing.

The terms “pancreatic insufficient” (PI) and “pancreatic sufficient” (PS), and the like, relate to whether a person has enough pancreatic function to grow and maintain health without supplemental pancreatic enzyme therapy (PERT) due to a failure to properly digest food because of a lack of digestive enzymes made by the pancreas.

As used herein, the terms “treat” and “treating” are not limited to the case where the subject (e.g., patient) is cured and the disease is eradicated. Rather, embodiments of the present disclosure also contemplate treatment that merely reduces symptoms, and/or delays disease progression.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other such that they are contained/circulating in the patient at the same time, e.g., considering half-lives.

A “cystic fibrosis drug” or “modulator” refers to a CFTR activator, corrector, potentiator, stabilizers, amplifiers, read-through agents, or combinations thereof. A “CFTR corrector” is a compound that acts by increasing the delivery and amount of functional CFTR protein to the cell surface, resulting in enhanced ion transport. Examples include elexacaftor, lumacaftor, tezacaftor. A “CFTR potentiator” is a compound that acts in the presence of endogenous or pharmacological CFTR activators to increase the channel gating activity of cell-surface localized CFTR, resulting in enhanced ion transport. An example is ivacaftor. As used herein, a “CFTR activator” is a compound that acts to stimulate CFTR-mediated ion transport, and includes agents that increase cAMP levels, such as b-adrenergic agonists, adenylate cyclase activators, and phosphodiesterase inhibitors. Other examples of CFTR activators include lubiprostone, unoprostone, or cobiprostone, forskolin, beta-2-agonists (such as albuterol and/or isoproterenol), genistein, pyrrolo[2,3-b]pyrazines derivatives (such as RP-107), 4-chlorobenzo[F]isoquinoline (CBIQ), 2-thioxo-4-amino-thiazoles (such as A01 and A02), 5-((Z)-2-(2-(allyloxy)phenyl)-1-cyanovinyl)-3-amino-1H-pyrazole-4-carbonitrile (Cact-A1).

CFTR function can be characterized at the cellular (in vitro) level using cell-based assays, such as an FRT assay (Van Goor, et al., (2009) PNAS 106(44); 18825-30; and Van Goor, et al. (2011) PNAS 108(46):18843-46) to measure the amount of chloride transport through the mutated CFTR channels. Residual function mutations result in a reduction of CFTR-dependent ion transport. For example, residual function mutations may result in more or less than about 10%, about 20%, about 30%, about 40%, about 50%, about 60%, about 70%, about 80% or about 90% reduction of CFTR activity in the FRT assay.

Any compound disclosed herein may be in a pharmaceutically acceptable salt thereof. As used herein, a “pharmaceutically acceptable salt” refers to any salt or salt of an ester of a compound of this disclosure that, upon administration to a recipient, is capable of providing, either directly or indirectly, a compound of this disclosure. Pharmaceutically acceptable salts are well known in the art. For example, pharmaceutically acceptable salts may include the FDA-approved commercially marketed salts: acetate, aluminum, benzenesulfonate, benzathine, benzoate, bicarbonate, bitartrate, bromide, calcium, calcium edetate, camsylate, carbonate, chloride, choline, citrate, diethanolamine, dihydrochloride, edetate, edisylate, esylate, ethylenediamine, fumarate, gluceptate, gluconate, glutamate, hydrobromide, hydrochloride, hydroxynaphthoate, iodide, isethionate, lactate, lactobionate, lithium, magnesium, malate, maleate, mandelate, meglumine, mesylate, nitrate, pamoate, pantothenate, phosphate/diphosphate, polygalacturonate, potassium, salicylate, sodium, stearate, subacetate, succinate, sulfate, tannate, tartrate, and zinc.

### Treatment of Bronchiectasis

Bronchiectasis is a complex, chronic respiratory condition, characterized by frequent cough and shortness of breath due to a range of conditions that include inherited mucociliary defects, inhalational airway injury, immunodeficiency states and prior respiratory infections. Bronchiectasis is characterized as a thickening and dilation of the walls of the bronchi from inflammation, infection, or other etiologies which result in the inability to clear mucus from the airway. Affected individuals are then more susceptible to repeated lung infections.

Bronchiectasis is commonly found in individuals with cystic fibrosis. However, non-cystic fibrosis bronchiectasis (NCFBE) is prevalent worldwide. These individuals are not considered to have a diagnosis of cystic fibrosis (CF) because they do not meet diagnostic criteria for CF. Some patients with NCFBE may have slightly elevated sweat chloride—but do not carry a diagnosis of CF or have known, disease-causing mutations in CFTR. They are generally believed to have a cause of bronchiectasis due to factors unrelated to mutations in CFTR.

CFTR modulators are not prescribed or approved for non-CF bronchiectasis and have not been considered to be of benefit for patients with NCFBE. This is because these compounds are highly personalized, specifically targeted molecules designed to activate mutant forms of CFTR. Trikafta™ is a combination of three CF drugs, elexacaftor, ivacaftor, and tezacaftor, that helps defective CFTR proteins work more effectively.

This disclosure relates to methods of treating bronchiectasis comprising administering an effective amount of a cystic fibrosis drug to a subject diagnosed with bronchiectasis (e.g., human patients with non-cystic fibrosis bronchiectasis (NCFBE)) and the subject is diagnosed with moderate elevated sweat chloride and pancreatic sufficiency. In certain embodiments, a sample of the subject is tested for presence of a known cystic fibrosis transmembrane conductance regulator mutation and no mutation is identified in the sample. In certain embodiments, moderate elevated sweat chloride is in a concentration between about 30 to 60 millimoles per liter (mEq/L). In certain embodiments, the subject has a normal sweat chloride is in a concentration of less than or about 30 mEq/L.

In certain embodiments, the subject is diagnosed to not have a F508del mutation in the cystic fibrosis transmembrane conductance regulator (CFTR) gene and/or not having any of the following mutations 3141del9, E822K, G1069R, L967S, R117L, S912L, 546insCTA, F191V, G1244E, L997F, R117P, S945L, A46D, F311del, G1249R, L1077P, R170H, S977F, A120T, F311L, G1349D, L1324P, R258G, S1159F, A234D, F508C, H139R, L1335P, R334L, S1159P, A349V, F508C, H199Y, L1480P, R334Q, S1251N, A455E, H939R, M152V, R347H, S1255P, A554E, F575Y, H1054D, M265R, R347L, T338I, A1006E, F1016S, H1085P, M952I, R347P, T1036N, A1067T, F1052V, H1085R, M952T, R352Q, T1053I, D110E, F1074L, H1375P, M1101K, R352W, V201M, D110H, F1099L, I148T, P5L, R553Q, V232D, D192G, G27R, I175V, P67L, R668C, V456A, D443Y, G85E, I336K, P205S, R751L, V456F, D443Y, R668C, G126D, I502T, P574H, R792G, V562I, D579G, G178E, I601F, Q98R, R933G, V754M, D614G, G178R, I618T, Q237E, R1066H, V1153E, D836Y, G194R, I807M, Q237H, R1070Q, V1240G, D924N, G194V, I980K, Q359R, R1070W, V1293G, D979V, G314E, I1027T, Q1291R, R1162L, W361R, D1152H, G463V, I1139V, R31L, R1283M, W1098C, D1270N, G480C, I1269N, R74Q, R1283S, W1282R, E56K, G551D, I1366N, R74W, S13F, Y109N, E60K, G551S, K1060T, D1270N, S341P, Y161D, E92K, G576A, L15P, S364P, Y161S, E116K, R668C, L165S, D1270N, S492F, Y563N, E193K, G622D, L206W, R75Q, S549N, Y1014C, E403D, G628R, L320V, R117C, S549R, Y1032C, E474K, G970D, L346P, R117G, S589N, E588V, G1061R, L453S, R117H, and S737F or other CFTR mutations known to cause clinical CF (cftr2.org).

In certain embodiments, a sample of the subject was tested for presence of a known cystic fibrosis transmembrane conductance regulator mutation and no mutation or only one mutation was identified in the sample, thereby providing a subject diagnosed without two known cystic fibrosis transmembrane conductance regulator mutations and carrying a diagnosis of non-cystic fibrosis bronchiectasis.

In certain embodiments, the subject for treatment is diagnosed with one copy of a CFTR mutation (i.e. heterozygote) optionally having moderate elevated sweat chloride is in a concentration between about 30 to 60 millimoles per liter (mEq/L).

In certain embodiments, this disclosure relates to methods of treating bronchiectasis comprising administering an effective amount of a cystic fibrosis drug to a subject diagnosed with non-CF bronchiectasis, wherein the subject is diagnosed as heterozygous for a CFTR disease causing mutation and not diagnosed as homozygous for any CFTR disease causing mutation and wherein the subject is not diagnosed with cystic fibrosis (CF). In certain embodiments the subject is diagnosed as having moderate elevated sweat chloride in a concentration between about 30 to 60 millimoles per liter (mEq/L).

In certain embodiments the subject is diagnosed as heterozygous for a single CFTR disease causing mutation selected from 3141del9, E822K, G1069R, L967S, R117L, S912L, 546insCTA, F191V, G1244E, L997F, R117P, S945L, A46D, F311del, G1249R, L1077P, R170H, S977F, A120T, F311L, G1349D, L1324P, R258G, S1159F, A234D, F508C, H139R, L1335P, R334L, S1159P, A349V, F508C, H199Y, L1480P, R334Q, S1251N, A455E, H939R, M152V, R347H, S1255P, A554E, F575Y, H1054D, M265R, R347L, T338I, A1006E, F1016S, H1085P, M952I, R347P, T1036N, A1067T, F1052V, H1085R, M952T, R352Q, T1053I, D110E, F1074L, H1375P, M1101K, R352W, V201M, D110H, F1099L, I148T, P5L, R553Q, V232D, D192G, G27R, I175V, P67L, R668C, V456A, D443Y, G85E, I336K, P205S, R751L, V456F, D443Y, R668C, G126D, I502T, P574H, R792G, V562I, D579G, G178E, I601F, Q98R, R933G, V754M, D614G, G178R, I618T, Q237E, R1066H, V1153E, D836Y, G194R, I807M, Q237H, R1070Q, V1240G, D924N, G194V, I980K, Q359R, R1070W, V1293G, D979V, G314E, I1027T, Q1291R, R1162L, W361R, D1152H, G463V, I1139V, R31L, R1283M, W1098C, D1270N, G480C, I1269N, R74Q, R1283S, W1282R, E56K, G551D, I1366N, R74W, S13F, Y109N, E60K, G551S, K1060T, D1270N, S341P, Y161D, E92K, G576A, L15P, S364P, Y161S, E116K, R668C, L165S, D1270N, S492F, Y563N, E193K, G622D, L206W, R75Q, S549N, Y1014C, E403D, G628R, L320V, R117C, S549R, Y1032C, E474K, G970D, L346P, R117G, S589N, E588V, G1061R, L453S, R117H, and S737F, and not have a disease causing mutation on the other allele and wherein the subject is not diagnosed with cystic fibrosis (CF).

In certain embodiments, the subject is not diagnosed to have a homozygous F508del mutation in the cystic fibrosis transmembrane conductance regulator (CFTR) gene and wherein the subject is not diagnosed with cystic fibrosis (CF).

In certain embodiments, the subject is not diagnosed to have a heterozygous F508del mutation in the cystic fibrosis transmembrane conductance regulator (CFTR) gene nor another CF mutation on the other allele and does not have CF, and wherein the subject is not diagnosed with cystic fibrosis (CF).

In certain embodiments, the subject is diagnosed to have a heterozygous F508del mutation in the cystic fibrosis transmembrane conductance regulator (CFTR) gene, and not another CF mutation on the other allele and wherein the subject is not diagnosed with cystic fibrosis (CF).

In certain embodiments, the cystic fibrosis drug is lumacaftor, elexacaftor, ivacaftor, tezacaftor, or combinations thereof. In certain embodiments, the cystic fibrosis drug is a combination of elexacaftor, ivacaftor, and tezacaftor. In certain embodiments, the cystic fibrosis drug is a combination of ivacaftor and lumacaftor. In certain embodiments, the cystic fibrosis drug is a combination of ivacaftor and tezacaftor. In certain embodiments, the cystic fibrosis drug is administered in combination with another active agent such as a bronchodilator, corticosteroid, antimuscarinic, antibiotic, or combinations thereof.

In certain embodiments, the drug is a candidate CFTR modulator such as phosphodiesterase inhibitors (PDE), PDE4 inhibitors, PDE5 inhibitors, ensifentrine, sildenafil, tadalafil, vardenafil, idrevloride, (2R,3S,4R,5R,6S)-5-amino-6-[(1R,2R,3S,4R,6S)-4,6-diamino-2-[(2S,3R,4S,5R)-5-[(1S)-1-aminoethyl]-3,4-dihydroxyoxolan-2-yl]oxy-3-hydroxycyclohexyl] oxy-2-[(1R)-1-hydroxyethyl]oxane-3,4-diol sulfuric acid (ELX-02), S-1226, MRT5005, cavosonstat, gallium 2-hydroxypropane-1,2,3-tricarboxylate (AR-501), VX-121, dirocaftor, Ajulemic acid, N-(3-carbamoyl-5,5,7,7-tetramethyl-4,7-dihydro-5H-thieno[2,3-c]pyran-2-yl)-1H-pyrazole-3-carboxamide (GLPG1837), galicaftor, lonodelestat, or combinations thereof.

In certain embodiments, the cystic fibrosis drug is administered as a pharmaceutical composition comprising the CF drug(s), or a pharmaceutically acceptable salt thereof. In certain embodiments, the cystic fibrosis drug a CFTR corrector, a CFTR potentiator, a CFTR activator, or combinations thereof. In certain embodiments, the cystic fibrosis drug is lumacaftor, elexacaftor, ivacaftor, tezacaftor, or combinations or salts thereof. In certain embodiments, the cystic fibrosis drug is a combination of elexacaftor, ivacaftor, and tezacaftor. In certain embodiments, the cystic fibrosis drug is a combination of ivacaftor and lumacaftor. In certain embodiments, the cystic fibrosis drug is a combination of ivacaftor and tezacaftor.

In certain embodiments, the cystic fibrosis drug is lumacaftor, elexacaftor, ivacaftor, tezacaftor, cavosonstat, olacaftor, posenacaftor, galicaftor, navocaftor, deutivacaftor, nesolicaftor, bamocaftor, ataluren, icenticaftor (QBW-251), N-(3-carbamoyl-5,5,7,7-tetramethyl-4,7-dihydro-5H-thieno[2,3-c]pyran-2-yl)-1H-pyrazole-5-carboxamide (GLPG-1837), (S)-3-amino-N-(2-hydroxypropyl)-5-((4-(trifluoromethoxy)phenyl)sulfonyl)picolinamide (GLPG-2451), N-(3-carbamoyl-5,5,7,7-tetramethyl-4,7-dihydro-5H-thieno[2,3-c]pyran-2-yl)-1H-pyrazole-3-carboxamide (ABBV 974), or combinations, or salts thereof.

In certain embodiments, this disclosure relates to the use of a pharmaceutical composition comprising cystic fibrosis drugs, or combinations or salts as reported herein, in the preparation of a medicament for the treatment of bronchiectasis.

In certain embodiments, this disclosure relates to the use of a pharmaceutical composition comprising cystic fibrosis drugs in the manufacture of a medicament for the treatment of bronchiectasis.

In certain embodiments, this disclosure contemplates a non-personalized use of CF drugs among patients with CF bronchiectasis, in particular those with sweat chloride elevation 30-60 mEq/L, and without a clinically approved genotype.

In certain embodiments, this disclosure contemplates uses in treating patients with non-CF bronchiectasis and a mildly elevated sweat chloride (i.e., suggesting diminished CFTR activity) not sufficient to establish a diagnosis of CF (but instead leading to a diagnosis of non-CF bronchiectasis) by administering CF drugs reported herein.

In certain embodiments, this disclosure contemplates uses in treating patients with non-CF bronchiectasis and normal sweat chloride (less than 30 mEq/L, normal CFTR function in the sweat gland) by administering CF drugs, provided activating the endogenous (and non-mutant) CFTR can enhance mucus clearance and/or minimize airway remodeling.

In certain embodiments, this disclosure contemplates uses in treating bronchiectasis in patients without disease-causing CFTR mutations which can nonetheless benefit from CF drugs.

In certain embodiments, a sample of the subject is tested for presence of a known cystic fibrosis transmembrane conductance regulator mutation and a mutation is found which is not identified as resulting in a defective cystic fibrosis transmembrane conductance regulator or a cause of CF, thereby contributing to a diagnosis of non-cystic fibrosis bronchiectasis (NCFBE). In certain embodiments, the mutation is a conservative substitution or non-conservative substitution inside or outside of a functional domain. In certain embodiments, the mutation is a conservative substitution or non-conservative substitution outside a functional domain or a conservative substitution inside a functional domain. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are valine-leucine-isoleucine, phenylalanine-tyrosine, lysine-arginine, alanine-valine, and asparagine-glutamine.

The human cystic fibrosis transmembrane conductance regulator has NCBI Reference Sequence number NP_000483.3 starting a position 1: MQRSPLEKAS VVSKLFFSWT RPILRKGYRQ RLELSDIYQI PSVDSADNLS EKLEREWDRE 61 LASKKNPKLI NALRRCFFWR FMFYGIFLYL GEVTKAVQPL LLGRIIASYD PDNKEERSIA 121 IYLGIGLCLL FIVRTLLLHP AIFGLHHIGM QMRIAMFSLI YKKTLKLSSR VLDKISIGQL 181 VSLLSNNLNK FDEGLALAHF VWIAPLQVAL LMGLIWELLQ ASAFCGLGFL IVLALFQAGL 241 GRMMMKYRDQ RAGKISERLV ITSEMIENIQ SVKAYCWEEA MEKMIENLRQ TELKLTRKAA 301 YVRYFNSSAF FFSGFFVVFL SVLPYALIKG IILRKIFTTI SFCIVLRMAV TRQFPWAVQT 361 WYDSLGAINK IQDFLQKQEY KTLEYNLTTT EVVMENVTAF WEEGFGELFE KAKQNNNNRK 421 TSNGDDSLFF SNFSLLGTPV LKDINFKIER GQLLAVAGST GAGKTSLLMV IMGELEPSEG 481 KIKHSGRISF CSQFSWIMPG TIKENIIFGV SYDEYRYRSV IKACQLEEDI SKFAEKDNIV 541 LGEGGITLSG GQRARISLAR AVYKDADLYL LDSPFGYLDV LTEKEIFESC VCKLMANKTR 601 ILVTSKMEHL KKADKILILH EGSSYFYGTF SELQNLQPDF SSKLMGCDSF DQFSAERRNS 661 ILTETLHRFS LEGDAPVSWT ETKKQSFKQT GEFGEKRKNS ILNPINSIRK FSIVQKTPLQ 721 MNGIEEDSDE PLERRLSLVP DSEQGEAILP RISVISTGPT LQARRRQSVL NLMTHSVNQG 781 QNIHRKTTAS TRKVSLAPQA NLTELDIYSR RLSQETGLEI SEEINEEDLK ECFFDDMESI 841 PAVTTWNTYL RYITVHKSLI FVLIWCLVIF LAEVAASLVV LWLLGNTPLQ DKGNSTHSRN 901 NSYAVIITST SSYYVFYIYV GVADTLLAMG FFRGLPLVHT LITVSKILHH KMLHSVLQAP 961 MSTLNTLKAG GILNRFSKDI AILDDLLPLT IFDFIQLLLI VIGAIAVVAV LQPYIFVATV 1021 PVIVAFIMLR AYFLQTSQQL KQLESEGRSP IFTHLVTSLK GLWTLRAFGR QPYFETLFHK 1081 ALNLHTANWF LYLSTLRWFQ MRIEMIFVIF FIAVTFISIL TTGEGEGRVG IILTLAMNIM 1141 STLQWAVNSS IDVDSLMRSV SRVFKFIDMP TEGKPTKSTK PYKNGQLSKV MIIENSHVKK 1201 DDIWPSGGQM TVKDLTAKYT EGGNAILENI SFSISPGQRV GLLGRTGSGK STLLSAFLRL 1261 LNTEGEIQID GVSWDSITLQ QWRKAFGVIP QKVFIFSGTF RKNLDPYEQW SDQEIWKVAD 1321 EVGLRSVIEQ FPGKLDFVLV DGGCVLSHGH KQLMCLARSV LSKAKILLLD EPSAHLDPVT 1381 YQIIRRTLKQ AFADCTVILC EHRIEAMLEC QQFLVIEENK VRQYDSIQKL LNERSLFRQA 1441 ISPSDRVKLF PHRNSSKCKS KPQIAALKEE TEEEVQDTRL (SEQ ID NO: 1).

Functional domains may include the membrane spanning domain 1 having amino acids of the transmembrane regions: 78 to 98, 123 to 146, 196 to 216, 223 to 243, 299 to 319, 340 to 358; nucleotide binding domain 1 having amino acids 389 to 670; a regulatory domain having amino acids 639 to 849; a membrane spanning domain 2 having amino acid of the transmembrane regions: 866 to 879, 919 to 939, 991 to 1011, 1014 to 1034, 1096 to 1116, 1131 to 1151; and a nucleotide binding domain 2 having amino acids 1208 to 1480 (PDZ-binding region amino acids 1478 to 1480).

In certain embodiments, the subject is diagnosed with a mutation outside or inside a functional domain with sweat chloride elevation 30-60 mEq/L, and without a clinically approved genotype. In certain embodiments, the subject is diagnosed with a heterologous mutation outside or inside a functional domain. In certain embodiments, the subject is diagnosed with a homologous mutation inside or outside a functional domain.

In certain embodiments, this disclosure contemplates treating patients with a diagnosis of bronchiectasis that have elevated sweat chloride, e.g., greater than 60 mEq/L, without a diagnosis of a clinically approved cystic fibrosis genotype, unable to identify a known cystic fibrosis transmembrane conductance regulator mutation, pancreatic insufficiency, or combinations thereof that respond to treatment with cystic fibrosis drugs such as lumacaftor, elexacaftor, ivacaftor, tezacaftor, or combinations thereof.

In certain embodiments, the cystic fibrosis drug is administered in combination with another active agent such as a bronchodilator, corticosteroid, antimuscarinic, antibiotic, or combinations thereof. In certain embodiments, the active agent is administered as a pill, an injection, a tablet, or syrup or using an inhaler or nebulizer compressed into a mist, optionally using an aqueous saline solution, inhaled through a mouthpiece or face mask.

In certain embodiments, the bronchodilator is a beta-2 agonist, such as salbutamol, salmeterol, formoterol and vilanterol or an anticholinergic, such as ipratropium, tiotropium, aclidinium, or glycopyrronium, or an antimuscarinic such as atropine or scopolamine, or theophylline. In certain embodiments, the cystic fibrosis drug is administered in combination with a bronchodilator albuterol, formoterol, or levalbuterol or salts thereof.

In certain embodiments, the cystic fibrosis drug is administered in combination with a mucolytic agent such as bromhexine or salts thereof.

In certain embodiments, the cystic fibrosis drug is administered in combination with an anti-inflammatory agent such as a corticosteroid, fluticasone, or salts thereof.

In certain embodiments, the cystic fibrosis drug is administered in combination with an antibiotic agent such as macrolides, azithromycin, antipseudomonal, fluoroquinolones, ciprofloxacin, levofloxacin, ceftazidime, piperacillin and tazobactam, imipenem, aminoglycosides, aztreonam, tobramycin, colistin, colistimethate sodium, or salt thereof.

In certain embodiments, the cystic fibrosis drug is administered in combination with a reversible inhibitor of dipeptidyl peptidase I (DPP1) such as brensocatib.

### Trikafta™ Treatment for Non-CF Bronchiectasis (NCFBE)

A patient carrying a diagnosis of CF who had no known mutations in CFTR but exhibited clinical features of cystic fibrosis including bronchiectasis and a modestly elevated sweat chloride, was treated with Trikafta™. This patient experienced a robust improvement in lung function following Trikafta™ therapy. The result indicated that a person with bronchiectasis, but no known CFTR mutations, may nonetheless respond strongly to Trikafta™. There are many patients with a diagnosis termed non-CF bronchiectasis. Such patients often have no known CFTR mutations but exhibit severe and life-threatening bronchiectasis. Bronchiectasis may be due to factors unrelated to mutations in CFTR. These individuals are not treated using Trikafta™ in part because they do not carry diagnostic CFTR mutations. Importantly, there is no effective molecular therapy available for NCFBE. The etiology of NCFBE is unknown and may be multi-factorial. It is intended that certain patients with non-CF bronchiectasis (and without diagnostic CFTR mutations) respond robustly to Trikafta™.

Experiments are performed to test clinical efficacy of a drug (Trikafta™) in patients with modestly elevated sweat chloride or pancreatic sufficiency. In vitro measurements of CFTR function may be performed in pluripotent stem cells reprogrammed to form airway epithelial monolayers.

What is claimed is:

1. A method of treating bronchiectasis comprising administering an effective amount of a cystic fibrosis drug to a subject diagnosed with non-CF bronchiectasis.

2. The method of claim 1, wherein the subject is diagnosed with pancreatic sufficiency.

3. The method of claim 1, wherein the subject is diagnosed with moderate elevated sweat chloride and the moderate elevated sweat chloride is in a concentration between 30 to 60 millimoles per liter.

4. The method of claim 1, wherein a sample of the subject was tested for presence of a known cystic fibrosis transmembrane conductance regulator mutation and no mutation was identified in the sample, thereby providing a subject diagnosed without a known cystic fibrosis transmembrane conductance regulator mutation.

5. The method of claim 1 wherein the subject is diagnosed to not have a F508del mutation in the cystic fibrosis transmembrane conductance regulator (CFTR) gene and not having any of the following mutations 3141del9, E822K, G1069R, L967S, R117L, S912L, 546insCTA, F191V, G1244E, L997F, R117P, S945L, A46D, F311del, G1249R, L1077P, R170H, S977F, A120T, F311L, G1349D, L1324P, R258G, S1159F, A234D, F508C, H139R, L1335P, R334L, S1159P, A349V, F508C, H199Y, L1480P, R334Q, S1251N, A455E, H939R, M152V, R347H, S1255P, A554E, F575Y, H1054D, M265R, R347L, T338I, A1006E, F1016S, H1085P, M952I, R347P, T1036N, A1067T, F1052V, H1085R, M952T, R352Q, T1053I, D110E, F1074L, H1375P, M1101K, R352W, V201M, D110H, F1099L, I148T, P5L, R553Q, V232D, D192G, G27R, I175V, P67L, R668C, V456A, D443Y, G85E, I336K, P205S, R751L, V456F, D443Y, R668C, G126D, I502T, P574H, R792G, V562I, D579G, G178E, I601F, Q98R, R933G, V754M, D614G, G178R, I618T, Q237E, R1066H, V1153E, D836Y, G194R, I807M, Q237H, R1070Q, V1240G, D924N, G194V, I980K, Q359R, R1070W, V1293G, D979V, G314E, I1027T, Q1291R, R1162L, W361R, D1152H, G463V, I1139V, R31L, R1283M, W1098C, D1270N, G480C, I1269N, R74Q, R1283S, W1282R, E56K, G551D, I1366N, R74W, S13F, Y109N, E60K, G551S, K1060T, D1270N, S341P, Y161D, E92K, G576A, L15P, S364P, Y161S, E116K, R668C, L165S, D1270N, S492F, Y563N, E193K, G622D, L206W, R75Q, S549N, Y1014C, E403D, G628R, L320V, R117C, S549R, Y1032C, E474K, G970D, L346P, R117G, S589N, E588V, G1061R, L453S, R117H, and S737F.

6. The method of claim 1, wherein the cystic fibrosis drug is lumacaftor, elexacaftor, ivacaftor, tezacaftor, or combinations thereof.

7. The method of claim 5, wherein the cystic fibrosis drug is a combination of elexacaftor, ivacaftor, and tezacaftor.

8. The method of claim 5, wherein the cystic fibrosis drug is a combination of ivacaftor and lumacaftor.

9. The method of claim 5, wherein the cystic fibrosis drug is a combination of ivacaftor and tezacaftor.

10. The method of claim 6 wherein the cystic fibrosis drug is administered in combination with another active agent such as a bronchodilator, corticosteroid, antimuscarinic, antibiotic, or combinations thereof.
